(19) United States
(12) Patent Application Publication
(10) Pub. No.: US 2002/0193624 A1
(43) Pub. Date: Dec. 19, 2002

(54) Preparation of alkylpolyalkylene glycol carboxylates

(75) Inventors: Michael Zirnstein, Schriesheim (DE); Ludwig Voelkel, Limburgerhof (DE); Matthias Kroner, Eisenberg (DE); Edward Bohres, Ludwigshafen (DE); Alfred Oftring, Bad Duerkheim (DE)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: BASF Aktiengesellschaft, Ludwigshafen (DE)

(21) Appl. No.: 10/166,143

(22) Filed: Jun. 11, 2002

(30) Foreign Application Priority Data
Jun. 18, 2001 (DE) 10129287.2

(51) Int. Cl. C07C 67/26; C07C 67/24

(52) U.S. Cl. 560/240

(57) ABSTRACT

Alkylpolyalkylene glycol carboxylates are prepared by a process which comprises reacting a carboxylic ester with an alkylene oxide in the presence of a multimetal cyanide compound of the formula I

M³_{z}M¹ₐ[M²(CN)_{b}(A)_{c}]_{d}.fM¹_{g}Xₙ.mM³ₚY_{q}.h(H₂O).eL.kP (I),

as catalyst and can be used as raw materials for concrete fluidizer polymers.

The present invention relates to a process for preparing alkylpolyalkylene glycol carboxylates from carboxylic esters and alkylene oxide, wherein the reaction is carried out using a multimetal cyanide compound as catalyst, and to the use of the alkylpolyalkylene glycol carboxylate prepared according to the present invention as raw materials for concrete fluidizer polymers.

Processes for preparing alkylpolyalkylene glycol carboxylates using catalysts are known per se. The type of catalyst used varies widely.

EP-A-0 140 545 describes a process for preparing glycol derivatives from epoxides and carboxylic esters using amidine catalysts.

Use is frequently made of modified hydrotalcites as catalysts. The use of calcined hydrotalcites as catalysts for preparing such alkoxylation products is disclosed in, for example, DE-A 19 843 384 or DE-A 3 914 131. DE-A 19 611 999 relates to a process for preparing alkoxylated alkyl esters of fatty acids using hydrotalcites modified with lithium hydroxides, alkaline earth metal salts or tin salts as catalysts.

DE-A 197 34 906 likewise describes a process for the alkoxylation of esters using modified hydrotalcites as catalyst. The catalysts described there are mixed hydroxides based on polycations.

Further catalysts used are basic mixed catalysts based on sodium and potassium hydroxides, oxides, carbonates, alkoxides or carboxylates. For example, JP 10 099 693 relates to an alkoxylation process which uses mixed catalysts based on basic alkali metal compounds or alkaline earth metal compounds and selected metal oxides.

The known processes suffer from disadvantages. Mention may be made, in particular, of a high residual content of the ester used as starting material and undesirable secondary reactions, for example the polymerization of unsaturated carboxylic esters due to the high alkoxylation temperatures required.

Metal cyanide compounds are known from the prior art as catalysts for polyadditions, in particular ring-opening polymerizations of alkylene oxides, as described, for example, in EP-A 0 892 002, EP-A 0 862 977 and EP-A 0 755 716.

WO 99/10407 describes a process for preparing polyethers having a hydroxy function and unsaturated groups. The synthesis is carried out by alkoxylation of an unsaturated monomer which bears reactive hydrogen atoms. The reaction is carried out in the presence of a double metal cyanide catalyst whose preparation is described in detail in U.S. Pat. No. 5,545,601.

It is an object of the present invention to provide suitable catalysts for the reaction of carboxylic esters with alkylene oxides, which catalysts enable the desired alkylpolyalkylene glycol carboxylates to be prepared under milder reaction conditions with avoidance of undesirable by-products.

We have found that this object is achieved by a process which uses a multimetal cyanide compound of the formula I as catalyst.

The present invention accordingly provides a process for preparing alkylpolyalkylene glycol carboxylates which comprises reacting at least one carboxylic ester with at least one alkylene oxide in the presence of a catalyst comprising a multimetal cyanide compound of the formula I:

M³_{z}M¹ₐ[M²(CN)_{b}(A)_{c}]_{d}.fM¹_{g}Xₙ.mM³ₚY_{q}.h(H₂O).eL.kP (I),

where

M¹ is at least one metal ion selected from the group consisting of Zn²⁺, Fe²⁺, Fe³⁺, CO³⁺, Ni²⁺, Mn²⁺, Co²⁺, Sn²⁺, Pb²⁺, Mo⁴⁺, Mo⁶⁺, Al³⁺, V⁴⁺, V⁵⁺, Sr²⁺, W⁴⁺, W⁶⁺, Cr²⁺, Cr³⁺, Cd²⁺, Hg²⁺, Pd²⁺, Pt²⁺, V²⁺, Mg², Ca²⁺, Ba²⁺, Cu²⁺.

M is at least one metal ion selected from the group consisting of Fe²⁺, F³⁺ CO²⁺ CO³⁺, Mn²⁺, Mn³⁺, V⁴⁺, V⁵⁺, Cr²⁺, Cr³⁺, Rh³⁺, R²⁺, Ir³⁺,

M¹ and M² are identical or different,

M³ is at least one metal ion selected from the group consisting of Li⁺, Na⁺, K⁺, Rb⁺, Cs⁺, Mg²⁺, Ca²⁺, Ba²⁺, Sr²⁺, ammonium ions of the formula R¹R²R³R⁴N⁺ where R¹, R², R³ and R⁴ are each H or a hydrocarbon radical having from 1 to 6 carbon atoms,

A, X and Y are each, independently of one another, an anion selected from the group consisting of halide, hydroxide, alkoxide, sulfate, carbonate, cyanide, thiocyanate, isocyanate, cyanate, carboxylate, oxalate, nitrate, nitrosyl, hydrogen sulfate, phosphate, dihydrogenphosphate, hydrogenphosphate and hydrogencarbonate,

L is a water-miscible ligand selected from the group consisting of alcohols, aldehydes, ketones, ethers, polyethers, esters, polyesters, polycarbonate, ureas, amides, primary, secondary and tertiary amines, ligands containing a pyridine nitrogen, nitriles, sulfides, phosphides, phosphites, phosphines, phosphonates and phosphates,

k is a fraction or integer greater than or equal to zero, and

P is an organic additive,

a, b, c, d, g, n, p, q and z are chosen so that the compound (I) is electrically neutral, and c or z or c and z can be 0,

e is the number of ligand molecules and is a fraction or integer greater than 0 or is 0,

f, k, h and m are each, independently of one another, a fraction or integer greater than 0 or 0.

Possible organic additives P are: polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylamide-co-maleic acid), polyacrylonitrile, polyalkyl acrylates, polyalkyl methacrylates, polyvinyl methyl ether, polyvinyl ethyl ether, polyvinyl acetate, polyvinyl alcohol, poly-N-vinylpyrrolidone, poly(N-vinylpyrrolidone-co-acrylic acid), polyvinyl methyl ketone, poly(4-vinylphenol), poly(acrylic acid-co-styrene), oxazoline polymers, polyalkylenimines, maleic acid and maleic anhydride copolymers, hydroxyethyl cellulose, polyacetates, ionic surface- and interfaceactive compounds, bile acids or their salts, esters or amides, carboxylic esters of polyhydric alcohols and glycosides.

The process of the present invention enables alkylpolyalkylene glycol carboxylates to be prepared formally in the manner of an insertion reaction by alkoxylation starting from carboxylic esters and alkylene oxides using a catalyst system according to the present invention in high yield and with complete conversion of the carboxylic esters.

For the purposes of the present invention, carboxylic esters without functional groups containing an active hydrogen, for example without hydroxy groups, can be used. However, it is likewise possible, according to the present invention, to use carboxylic esters bearing functional groups containing an active hydrogen as long as this does not result in undesirable secondary reactions. Furthermore, carboxylic esters bearing various functional groups containing active hydrogen or without active hydrogen can also be used for the purposes of the present invention.

In a preferred embodiment, the invention provides a process for preparing alkylpolyalkylene glycol carboxylates in which one or more of the following conditions are fulfilled:

(A) M¹ is selected from the group consisting of Zn²⁺, Fe²⁺, Fe³⁺, Co³⁺, Ni²⁺, Mn²⁺, Co²⁺

(B) M² is selected from the group consisting of Fe²⁺, Fe³⁺, Co³⁺,

(C) M³ is selected from the group consisting of Na⁺, K⁺, Cs⁺, ammonium ions of the formula R¹R²R³R⁴N⁺.

For the purposes of the present invention further preference is given to at least M¹ or M² being Fe²⁺or Fe³⁺, in particular together with the other preferred metal ions specified under (A) to (C).

In another preferred embodiment of the present invention, both M¹ and M² are Fe²⁺ or Fe³⁺, in particular together with the other preferred metal ions specified for M³ under(C).

Examples of multimetal cyanide compounds which are suitable for the purposes of the present invention are compounds of the formula II:

M^{a}Fe[Fe(CN)₆]nH₂O (II),

where M^{a} can be potassium (K⁺) or ammonium (NH₄⁺), with K⁺ being preferred over NH₄⁺ for the purposes of the present invention.

According to the present invention, the catalyst used can also comprise compounds of the formula III:

Fe₄[Fe(CN)₆]₃ (III)

in which further ions as defined in respect of the formula (I) may also be present in place of from one to three Fe(III) ions.

Multimetal cyanide compounds which have been found to be particularly useful as catalyst for the purposes of the present invention are, for example, the following iron hexacyanoferrates, iron blue pigments, iron cyanide blue, Vossen-Blau®, Prussian blue, Berlin blue, Turnbull's blue, Milori blue, Paris blue. Likewise suitable are, for example, Panax Blue®, Manox Blau® or Sicomet® Blau.

The use according to the present invention of the multimetal cyanide compounds enables the desired alkylpolyalkylene glycol carboxylates to be prepared by means of an alkoxylation reaction under mild reaction conditions.

The multimetal cyanide compounds are generally produced by reaction of at least one metal salt with at least one cyanometalic compound. Cyanometalic compounds which can be used are, for example, salts or acids. In the process of the present invention, contamination by alkali metal or alkaline earth metal salts does not interfere, so that complicated and costly purification of the catalysts becomes unnecessary.

In a preferred embodiment, the invention provides a process for preparing alkylpolyalkylene glycol carboxylates in which the multimetal cyanide compound used as catalyst is crystalline, partially crystalline, amorphous or partially amorphous.

It is possible according to the invention for a catalyst precursor compound to be prepared first and then to be converted, for example by oxidation, reduction, recrystallization or other reactions, into the actual catalytically active compound. Thus, for example, it is also conceivable according to the present invention for the precursor compound to be introduced into the reaction and the actual catalytically active compound to be generated only in the reaction medium in the presence of the components to be reacted.

It is also possible for the morphology of the multimetal cyanide particles to be controlled by addition of suitable substances, for example surface-active substances, so as to achieve an increased activity for the reaction to be catalyzed.

For the purposes of the present invention, the amount of catalyst used is from 0.001 to 30% by weight, preferably from 0.01 to 10% by weight, particularly preferably from 0.1 to 5% by weight or from 0.2 to 3% by weight, in each case based on the amount of carboxylic ester used.

The invention therefore provides, in particular, a process for preparing alkylpolyalkylene glycol carboxylates in which the catalyst is used in an amount of from 0.01 to 30% by weight, based on the amount of the carboxylic ester used.

In principle, esters of all substituted and unsubstituted branched or unbranched carboxylic acids can be used in the process of the present invention, as long as the functional groups of the carboxylic ester do not adversely affect the reaction being catalyzed.

For the purposes of the present invention, the alkyl component of the carboxylic ester is, in particular, a branched or unbranched saturated alkyl radical having from 1 to 22 carbon atoms or a branched or unbranched, monounsaturated or polyunsaturated alkyl radical having from 2 to 22 carbon atoms, particularly preferably a methyl radical.

For the purposes of the present invention, preference is given to using esters of the following carboxylic acids: substituted or unsubstituted, saturated or unsaturated monocarboxylic acids having from 3 to 22 carbon atoms, substituted or unsubstituted, saturated dicarboxylic acids having from 2 to 36 carbon atoms, substituted or unsubstituted, unsaturated dicarboxylic acids having from 4 to 36 carbon atoms and substituted or unsubstituted aromatic monocarboxylic and dicarboxylic acids.

In a further embodiment, the invention accordingly provides a process for preparing alkylpolyalkylene glycol carboxylates in which the carboxylic ester is derived from carboxylic acids selected from the group consisting of substituted or unsubstituted, saturated or unsaturated monocarboxylic acids having from 3 to 22 carbon atoms, substituted or unsubstituted, saturated dicarboxylic acids having from 2 to 36 carbon atoms, substituted or unsubstituted, unsaturated dicarboxylic acids having from 4 to 36 carbon atoms and substituted or unsubstituted aromatic monocarboxylic and dicarboxylic acids.

Carboxylic esters which are particularly preferred according to the present invention are ones which are derived from the following carboxylic acids: unsaturated substituted or unsubstituted monocarboxylic acids having from 3 to 5 carbon atoms and unsaturated substituted or unsubstituted dicarboxylic acids having from 4 to 8 carbon atoms, for example acrylic acid, methacrylic acid or crotonic acid, fumaric acid, maleic acid or itaconic acid; saturated substituted or unsubstituted monocarboxylic acids having from 1 to 5 carbon atoms and saturated substituted or unsubstituted dicarboxylic acids having from 2 to 5 carbon atoms, for example formic acid, acetic acid, propionic acid, pivalic acid, oxalic acid, malonic acid or succinic acid.

Furthermore, preference is also given to saturated or unsaturated substituted or unsubstituted monocarboxylic acids having from 6 to 22 carbon atoms which may also contain cycloaliphatic structural elements, for example hexanoic acid, heptanoic acid, cyclohexanecarboxylic acid, 2-ethylhexanoic acid, capric acid (C10), myristic acid (C14), palmitic acid (C16), stearic acid (C18), oleic acid, behenic acid (C22); saturated or unsaturated substituted or unsubstituted dicarboxylic acids having from 6 to 36 carbon atoms which, in particular, contain cycloaliphatic structural elements, for example adipic acid, pimelic acid (C7), azelaic acid (C9), sebacic acid (C10), dimeric fatty acids having 36 carbon atoms; substituted or unsubstituted aromatic monocarboxylic and dicarboxylic acids, for example benzoic acid, phthalic acid, isophthalic acid, terephthalic acid or naphthalenecarboxylic acids.

Very particular preference is given to using acrylic and methacrylic esters. The invention therefore provides, in a preferred embodiment, a process for preparing alkylpolyalkylene glycol carboxylates in which the carboxylic ester is methyl acrylate or methyl methacrylate.

In the process of the present invention, it is in principle possible to use all alkylene oxides which are known to those skilled in the art. For example, substituted or unsubstituted alkylene oxides having from 2 to 24 carbon atoms, preferably alkylene oxides having halogen, hydroxy, acyclic ether or ammonium substituents, are used. Particular mention may be made of: aliphatic 1,2-alkylene oxides having from 2 to 4 carbon atoms, for example ethylene oxide, propylene oxide, 1,2-butylene oxide, 2,3-butylene oxide or isobutylene oxide, aliphatic 1,2-alkylene oxides having from 5 to 24 carbon atoms, cycloaliphatic alkylene oxides, for example cyclopentene oxide, cyclohexene oxide or 1,5,9-cyclododecatriene monoxide, araliphatic alkylene oxides, for example styrene oxide.

Examples of preferred substituted alkylene oxides are epichlorohydrin, epibromohydrin, 2,3-epoxy-1-propanol, 1-allyloxy-2,3-epoxypropane, 2,3-epoxypropylphenyl ether, 2,3-epoxypropyl isopropyl ether, 2,3-epoxypropyl octyl ether or 2,3-epoxypropyltrimethylammonium chloride.

Particular preference is given to using 1,2-alkylene oxides having from 2 to 4 carbon atoms, in particular ethylene oxide or propylene oxide, in the process of the present invention.

In a preferred embodiment, the invention therefore provides a process for preparing alkyl polyalkylene glycol carboxylates from a carboxylic acid esters and an alkylene oxide, wherein the alkylene oxide is a 1,2-alkylene oxide having from 2 to 4 carbon atoms.

Particular preference is given to using ethylene oxide or propylene oxide as alkylene oxide.

For the purposes of the present invention, the carboxylic ester and the alkylene oxide are used in a ratio of from 1:1 to 1:200, preferably in a ratio from 1:5 to 1:100 and particularly preferably in a ratio from 1:10 to 1:50.

The reaction of the carboxylic ester with the alkylene oxide in the process of the present invention can be carried out at from 20 to 200° C. Preference is given to a temperature range from 40 to 150° C., in particular from 50 to 120° C. The reaction can be carried out either at atmospheric pressure or at subatmospheric pressure, and also at superatmospheric pressure, for example at a pressure of from 0.8 to 50 bar, in particular at a pressure of from 1 to 10 bar.

The invention therefore also provides a process for preparing alkylpolyalkylene glycol carboxylates in which the temperature during the reaction of the carboxylic ester with the alkylene oxide is from 50 to 120° C.

In a further embodiment, the invention provides a process for preparing alkylenepolyalkylene glycol carboxylates in which the pressure during the reaction of the carboxylic ester with the alkylene oxide is from 1 to 10 bar.

In the process of the present invention, the reaction can be carried out batchwise or continuously. It can be carried out in a stirred reactor, a tube reactor, a loop reactor, a fixed-bed reactor or a moving-bed reactor.

Auxiliaries and additives known to those skilled in the art can be added in the reaction of the carboxylic ester with the alkylene oxide. In a preferred embodiment, the reaction of the carboxylic ester with the alkylene oxide is carried out in the presence of at least one polymerization inhibitor. Examples of polymerization inhibitors which can be used are hydroquinone, hydroquinone monomethyl ether, 2,5-di-t-butylhydroquinone, 2,6-di-t-butyl-p-cresol, nitroso compounds such as isoacryloyl nitrite, nitrosodiphenylamine or Nnitrosocyclohexyldroxylamine, methylene blue, phenothiazine, tannic acid and diphenylamine. It is also possible, for the purposes of the present invention, to use two or more of these polymerization inhibitors. The polymerization inhibitors are used in amounts of from 10 to 50,000 ppm, in particular from 100 to 10,000 ppm, in each case based on the carboxylic ester used.

Furthermore, small amounts of molecular oxygen or nitrogen monoxide which do not pose safety problems can additionally be used in the process of the present invention.

It is not necessary to use solvents for the reaction of the carboxylic ester with the alkylene oxide in the process of the present invention. However, it is likewise possible to carry out the process of the present invention in the presence of water or organic solvents such as aliphatic, cycloaliphatic or aromatic hydrocarbons, alkyls, ethers, acetals, ketones, esters or cyclic carbonates.

According to the present invention, the catalyst can be separated from the reaction product by, for example, filtration, in particular deep bed filtration, crossflow filtration, membrane filtration or ultrafiltration.

The alkylpolyalkylene glycol carboxylates prepared according to the present invention can be used, for example, as monomers for free-radical homopolymerization or copolymerization reactions. These homopolymers or copolymers can be used, for example, as concrete fluidizer polymers. The present invention therefore also provides for the use of the alkylpolyalkylene glycol carboxylates prepared according to the present invention as raw materials for concrete fluidizer polymers.

The invention is illustrated by the examples below.

### EXAMPLES

### Example 1

### Catalyst Preparation

180 g of a 30% strength aqueous solution of iron(III) chloride hexahydrate were added dropwise to 211 g of a 30% strength aqueous solution of potassium hexacyanoferrate(II) trihydrate while stirring. The mixture was stirred for another half hour and subsequently filtered with suction. The filter residue was washed twice with methanol by stirring a corresponding slurry for 30 minutes in each case and then filtering with suction. The solid was dried at 50° C. under reduced pressure. This gave 77.4 g of a dark blue powder.

Elemental analysis of the product obtained indicated the following composition: C: 18.5%, H: 1.4%, N: 20.0%, O: 9.4%, Fe: 30.0%, K: 12.2%, Cl: 8.2%

### Example 2

### Alkoxylation Reaction

4.8 g of phenothiazine were dissolved in 120 g of methyl methacrylate, and 4.8 g of the catalyst prepared as described in Example 1 were suspended in the solution. This mixture was treated with 866 g of ethylene oxide gas at 80° C. in a pressure reactor, with the pressure rising to a maximum of 8.0 atm. The mixture was stirred for 18 hours while maintaining the reactor jacket temperature in the range from 75 to 81° C. The pressure dropped to 3.8 atm during this time. This gave 922 g of crude product from which residual ethylene oxide was removed under reduced pressure. The product crystallized on cooling to give a wax-like solid.

The product had a very narrow molar mass distribution, with an average of 14 ethylene oxide molecules having been inserted into the ester bond. No methacrylic ester could be detected, i.e. the conversion of the methacrylic ester was complete.

We claim:

1. A process for preparing alkylpolyalkylene glycol carboxylates which comprises reacting at least one carboxylic ester with at least one alkylene oxide in the presence of a catalyst comprising a multimetal cyanide compound of the formula I:
M³_{z}M¹ₐ[M²(CN)_{b}(A)_{c}]_{d}.fM¹_{g}Xₙ.mM³ₚY_{q}.h(H₂O).eL.kP (I),
where
M¹ is at least one metal ion selected from the group consisting of Zn²⁺, Fe²⁺, Fe³⁺, CO³⁺, Ni²⁺, Mn²⁺, Co²⁺, Sn²⁺, Pb²⁺, Mo⁴⁺, Mo⁶⁺, Al³⁺, V⁴⁺, V⁵⁺, Sr²⁺, W⁴⁺, W⁶⁺, Cr²⁺, Cr³⁺, Cd²⁺, Hg²⁺, Pd²⁺, Pt²⁺, V²⁺, Mg²⁺, Ca²⁺, Ba²⁺, Cu²⁺.
M² is at least one metal ion selected from the group consisting of Fe²⁺, Fe³⁺ Co²⁺ Co³⁺, Mn²⁺, Mn³⁺, V⁴⁺, V⁵⁺, Cr²⁺, Cr³⁺, Rh³⁺, Ru²⁺, Ir³⁺,
M¹ and M² are identical or different,
M³ is at least one metal ion selected from the group consisting of Li⁺, Na⁺, K⁺, Rb⁺, Cs⁺, Mg²⁺, Ca²⁺, Ba²⁺, Sr²⁺, ammonium ions of the formula R¹R²R³R⁴N⁺ where R¹, R², R³ and R⁴ are each H or a hydrocarbon radical having from 1 to 6 carbon atoms,
A, X and Y are each, independently of one another, an anion selected from the group consisting of halide, hydroxide, alkoxide, sulfate, carbonate, cyanide, thiocyanate, isocyanate, cyanate, carboxylate, oxalate, nitrate, nitrosyl, hydrogen sulfate, phosphate, dihydrogenphosphate, hydrogenphosphate and hydrogencarbonate,
L is a water-miscible ligand selected from the group consisting of alcohols, aldehydes, ketones, ethers, polyethers, esters, polyesters, polycarbonate, ureas, amides, primary, secondary and tertiary amines, ligands containing a pyridine nitrogen, nitriles, sulfides, phosphides, phosphites, phosphines, phosphonates and phosphates,
k is a fraction or integer greater than or equal to zero, and
P is an organic additive,
a, b, c, d, g, n, p, q and z are chosen so that the compound (I) is electrically neutral, and c or z or c and z can be 0,
e is the number of ligand molecules and is a fraction or integer greater than 0 or is 0,
f, k, h and m are each, independently of one another, a fraction or integer greater than 0 or 0.

2. A process for preparing alkylpolyalkylene glycol carboxylates as claimed in claim 1, wherein one or more of the following conditions are fulfilled:
(A) M¹ is selected from the group consisting of Zn²⁺, Fe²⁺, Fe²⁺, Co²⁺, Ni²⁺, Mn²⁺, Co²⁺
(B) M² is selected from the group consisting of Fe²⁺, Fe³⁺, Co³⁺,
(C) M³ is selected from the group consisting of Na⁺, K⁺, ammonium ions of the formula R¹R²R³R⁴N⁺.

3. A process for preparing alkylpolyalkylene glycol carboxylates as claimed in claim 1, wherein M¹ or M² is Fe²⁺ or Fe³⁺.

4. A process for preparing alkylpolyalkylene glycol carboxylates as claimed in claim 2, wherein M¹ or M² is Fe²⁺ or Fe³⁺.

5. A process for preparing alkylpolyalkylene glycol carboxylates as claimed in claim 1, wherein both M¹ and M² are Fe²⁺ or Fe³⁺.

6. A process for preparing alkylpolyalkylene glycol carboxylates as claimed in claim 2, wherein both M¹ and M² are Fe²⁺ or Fe³⁺.

7. A process for preparing alkylpolyalkylene glycol carboxylates as claimed in claim 1, wherein the multimetal cyanide compound is crystalline.

8. A process for preparing alkylpolyalkylene glycol carboxylates as claimed in claim 2, wherein the multimetal cyanide compound is crystalline.

9. A process for preparing alkylpolyalkylene glycol carboxylates as claimed in claim 1, wherein the catalyst is used in an amount of from 0.01 to 30% by weight, based on the amount of the carboxylic ester.

10. A process for preparing alkylpolyalkylene glycol carboxylates as claimed in claim 2, wherein the catalyst is used in an amount of from 0.01 to 30% by weight, based on the amount of the carboxylic ester.

11. A process for preparing alkylpolyalkylene glycol carboxylates as claimed in claimn 1, wherein the carboxylic ester is methyl acrylate or methyl methacrylate.

12. A process for preparing alkylpolyalkylene glycol carboxylates as claimed in claim 2, wherein the carboxylic ester is methyl acrylate or methyl methacrylate.

13. A process for preparing alkylpolyalkylene glycol carboxylates as claimed in claim 1, wherein the alkylene oxide is ethylene oxide or propylene oxide.

14. A process for preparing alkylpolyalkylene glycol carboxylates as claimed in claim 2, wherein the alkylene oxide is ethylene oxide or propylene oxide.

15. A process for preparing alkylpolyalkylene glycol carboxylates as claimed in claim 1, wherein the temperature during the reaction is from 50 to 120° C. or the pressure during the reaction of the carboxylic ester with the alkylene oxide is from 1 to 10 bar or both.

16. A process for preparing alkylpolyalkylene glycol carboxylates as claimed in claim 2, wherein the temperature during the reaction is from 50 to 120° C. or the pressure during the reaction of the carboxylic ester with the alkylene oxide is from 1 to 10 bar or both.

17. The method of using an alkylpolyalkylene glycol carboxylate prepared by a process as claimed in claim 1 as raw material for concrete fluidizer polymers.

18. The method of using an alkylpolyalkylene glycol carboxylate prepared by a process as claimed in claim 2 as raw material for concrete fluidizer polymers.

19. The method of using an alkylpolyalkylene glycol carboxylate prepared by a process as claimed in claim 13 as raw material for concrete fluidizer polymers.

20. The method of using an alkylpolyalkylene glycol carboxylate prepared by a process as claimed in claim 14 as raw material for concrete fluidizer polymers.
